(19) United States
(12) Patent Application Publication
(10) Pub. No.: US 2024/0100053 A1
(43) Pub. Date: Mar. 28, 2024

(54) PHARMACEUTICAL COMPOSITION

(71) Applicant: TOKYO METROPOLITAN GERIATRIC HOSPITAL AND INSTITUTE OF GERONTOLOGY, Tokyo (JP)

(72) Inventors: Toshiyuki ISHIWATA, Tokyo (JP); Norihiko SASAKI, Tokyo (JP)

(73) Assignee: TOKYO METROPOLITAN GERIATRIC HOSPITAL AND INSTITUTE OF GERONTOLOGY, Tokyo (JP)

(21) Appl. No.: 18/260,974

(22) PCT Filed: Jan. 13, 2022
(86) PCT No.: PCT/JP2022/000890

§ 371 Date: Nov. 7, 2023

(30) Foreign Application Priority Data
Jan. 13, 2021 (JP) 2021-003557

(51) Int. Cl. A61K 31/517; A61K 31/353; A61K 47/68; A61P 35/00; C07K 16/28

(52) U.S. Cl. CPC A61K 31/517; A61K 31/353; A61K 47/6845; A61P 35/00; C07K 16/2863; C07K 2317/24

(57) ABSTRACT

The purpose of the present invention is to provide an effective therapeutic pharmaceutical composition for treating pancreatic cancer. The problem can be solved by a pharmaceutical composition comprising a fibroblast growth factor receptor 4 inhibitor and a senolytic drug as active ingredients.

### TECHNICAL FIELD

The present invention relates to a pharmaceutical composition. According to the present invention, pancreatic cancer can be effectively treated.

### BACKGROUND ART

Pancreatic cancer is rapidly increasing, especially among the elderly, and many cases are inoperable because the cancer has already invaded or metastasized at the time of detection. Thus, the situation remains serious, with less than 10% of patients surviving after five years. Therefore, there is a need for early diagnosis of pancreatic cancer and development of new treatment methods.

It has been reported that signal transduction through fibroblast growth factor receptor 4 (FGFR4), a specific receptor for fibroblast growth factor 19 (FGF19), is associated with the development and progression of pancreatic cancer (Non-Patent Literature 1).

### CITATION LIST

### Non-Patent Literature

[NON-PATENT LITERATURE 1] International Journal of Oncology (Greece) 2011, Vol. 38, p. 133-143

### SUMMARY OF INVENTION

### Technical Problem

However, research on the treatment of pancreatic cancer has not progressed. Therefore, the purpose of the present invention is to provide an effective therapeutic pharmaceutical composition for treating pancreatic cancer.

### Solution to Problem

The present inventors have conducted intensive studies into an effective therapeutic pharmaceutical composition for treating pancreatic cancer, as a result, surprisingly found that the pharmaceutical composition exhibiting an excellent therapeutic effect to pancreatic cancer can be obtained by using fibroblast growth factor receptor 4 inhibitor and senolytic drug as active ingredients.

The present invention is based on the above findings.

Accordingly, the present invention relates to:
[1] a pharmaceutical composition comprising a fibroblast growth factor receptor 4 inhibitor and a senolytic drug as active ingredients,
[2] the pharmaceutical composition of item [1], wherein the fibroblast growth factor receptor 4 inhibitor is roblitinib, BLU-554, or BLU9931,
[3] the pharmaceutical composition of item [1] or [2], wherein the senolytic drug is quercetin,
[4] the pharmaceutical composition according to any one of items [1] to [3], for treating pancreatic cancer,
[5] a pharmaceutical composition for treating pancreatic cancer, comprising a fibroblast growth factor receptor 4 immune complex in which an anticancer agent or a photosensitive substance is conjugated to an antibody specific for fibroblast growth factor receptor 4,
[6] the pharmaceutical composition for treating pancreatic cancer of item [5], wherein the photosensitive substance is phthalocyanine,
[7] the pharmaceutical composition for treating pancreatic cancer of item [5] or [6], wherein the antibody is a humanized antibody, and
[8] a method for determining an effect of treatment of the pharmaceutical composition for treating pancreatic cancer according to any one of items [5] to [7].

The present specification discloses:
[9] a method for treating pancreatic cancer characterized in that a therapeutic effective amount of fibroblast growth factor receptor 4 inhibitor is administered to a pancreatic cancer patient,
[10] a method for treating pancreatic cancer of item [9], characterized in that a therapeutic effective amount of senolytic drug is further administered to the pancreatic cancer patient,
[11] a fibroblast growth factor receptor 4 inhibitor for a use in a method for treating pancreatic cancer,
[12] a fibroblast growth factor receptor 4 inhibitor and a senolytic drug, for a use in a method for treating pancreatic cancer,
[13] use of a fibroblast growth factor receptor 4 inhibitor for preparing a pharmaceutical composition for treating pancreatic cancer, and
[14] use of a fibroblast growth factor receptor 4 inhibitor and a senolytic drug for preparing a pharmaceutical composition for treating pancreatic cancer.

### Advantageous Effects of Invention

According to the present invention, pancreatic cancer patients can be effectively treated.

### BRIEF DESCRIPTION OF THE DRAWINGS

FIG. 1 is a schematic diagram showing a therapeutic mechanism of the pharmaceutical compositions of the present invention.

FIG. 2 is a schematic diagram showing an action of FGFR4 inhibitors in FGFR4-positive pancreatic cancer cells.

FIG. 3 is figures showing effects of BLU9931 on the inhibition of cell proliferation (A), and cytotoxicity (B) and cell cycle (C) in FGFR4-high expressing PK-1 cells and FGFR4-low expressing PK-45P cells.

FIG. 4 is photographs and graphs of immunoblots showing that expression of ERK, AKT, and STAT3 downstream of FGF19/FGFR4 signaling are suppressed by BLU9931.

FIG. 5 is photographs and a graph showing that BLU9931 inhibits cell invasion.

FIG. 6 is photographs (A) and a graph (B) showing aging of pancreatic cancer cells by BLU9931.

FIG. 7 is micrographs and a graph showing DNA damage after 3 days and 7 days of incubation with BLU9931.

FIG. 8 is graphs showing survival rates of pancreatic cancer cells treated with an FGFR4 inhibitor (BLU9931) and a senolytic drug (quercetin (A) or dasatinib (B)).

### DESCRIPTION OF EMBODIMENTS

### [1] Pharmaceutical Composition

The pharmaceutical composition of the present invention comprises a fibroblast growth factor receptor 4 inhibitor and a senolytic drug as active ingredients.

### Fibroblast Growth Factor Receptor 4

There are four known types of fibroblast growth factor receptors (hereinafter sometimes referred to as FGFRs): FGFR1, FGFR2, FGFR3, and FGFR4. FGFRs are composed of three extracellular immunoglobulin-like domains (D1-3), a transmembrane domain, and an intramolecular domain with tyrosine kinase activity. Among the above four FGFR1-4, FGFR4 is a target of the pharmaceutical composition of the present invention. FGFR4 is rarely expressed in normal human pancreatic tissues, but is abundantly expressed in cancer cells of patients with advanced stage pancreatic cancer as shown in the examples below.

The specific ligand of FGFR4 is fibroblast growth factor 19 (hereinafter sometimes referred to as FGF19).

### Pancreatic Cancer

More than 90% of pancreatic cancers are pancreatic ductal adenocarcinoma formed in the cells of the pancreatic duct, and the term “pancreatic cancer” usually refers to these pancreatic duct cancers. Cancer of the pancreas includes neuroendocrine tumors, intraductal papillary mucinous tumors, or the like. Risk factors for pancreatic cancer include chronic pancreatitis, diabetes, obesity, and smoking.

The pancreatic cancer targeted by the pharmaceutical composition of the present invention is a pancreatic cancer that expresses FGFR4. The pancreatic cancer expressing FGFR4 is not limited. However, they may have large tumor size and advanced stage.

The stage of pancreatic cancer progression includes a size and extent of the primary tumor indicated by T1-T4, wherein the pancreatic cancer is classified into T1 (cancer has less than 2 cm of tumor diameter and confined to the pancreas), T2 (cancer has greater than 2 cm of tumor diameter and confined to the pancreas), T3 (cancer has invaded intra-pancreatic bile duct, duodenum, or peripancreatic tissues), and T4 (cancer invasion into the adjacent T4 (cancer has invaded adjacent large blood vessels, extrapancreatic nerve plexus, or other organs).

Further, in the classification by lymph node metastasis, the pancreatic cancer is classified into N0 (lymph node metastasis (−)), N1 (metastasis to group 1 lymph nodes only (+)), N2 (metastasis to group 2 lymph nodes (+)), and N3 (metastasis to group 3 lymph nodes (+)).

Furthermore, in the classification by distant metastasis, the pancreatic cancer is classified into MO (no distant metastasis) and M1 (distant metastasis is detected).

By combining these classifications, pancreatic cancer is classified into stage 0, IA, IB, IIA, IIB, III, and IV, as shown in Table 1 below.

TABLE 1

| | | | |
|---|---|---|---|
| Stage 0 | T0, Tis | N0 | M0 |
| Stage IA | T1(T1a, T1b, T1c) | N0 | M0 |
| Stage IB | T2 | N0 | M0 |
| Stage IIA | T3 | N0 | M0 |
| Stage IIB | T0, Tis, T1(T1a, T1b, T1c), T2, T3 | N1(N1a, N1b) | M0 |
| Stage III | T4 | Any N | M0 |
| Stage IV | Any T | Any N | M1 |

### Fibroblast Growth Factor Receptor 4 Inhibitor

Fibroblast growth factor receptor 4 inhibitors (hereinafter sometimes referred to as FGFR4 inhibitors) included in the pharmaceutical compositions of the present invention are not limited, as long as it can inhibit the function of FGFR4, but include BLU9931, BLU-554, or roblitinib.

Fibroblast growth factor receptor 4 inhibitors promote aging of pancreatic cancer cells expressing FGFR4, but not limited thereto. For example, they may promote the expression of SA-β-Gal, which is expressed in aging cells. Further, they may also accelerate aging of FGFR4-expressing cells by damaging their DNA.

Fibroblast growth factor receptor 4 inhibitor alone can inhibit the invasion of pancreatic cancer cells in vivo and suppress the progression of pancreatic cancer.

BLU9931 is a compound represented by the following formula (1):

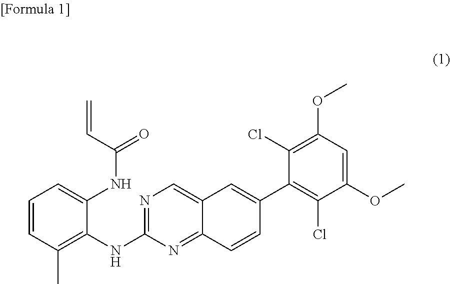 [SMILES: C=CC(=O)Nc1cccc(C)c1Nc1ncc2cc(-c3c(Cl)c(OC)cc(OC)c3Cl)ccc2n1]

It does not inhibit FGFR1-3, but selectively inhibits FGFR4. In FGFR1-3, the 552nd amino acid of hinge 1 is tyrosine (Y), whereas in FGFR4, the 552nd amino acid of hinge 1 is cysteine (C). BLU9931 is thought to selectively inhibit FGFR4 by binding to this cysteine.

Roblitinib is a compound represented by the following formula (2):

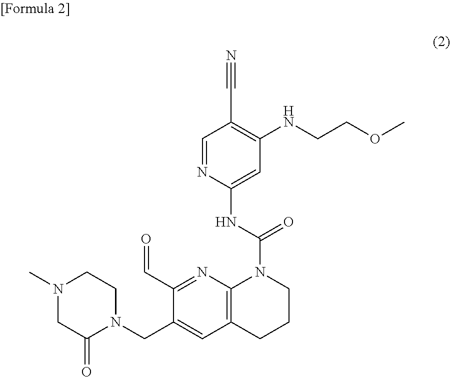

### Senolytic Drug

Senolytic drugs included in the pharmaceutical compositions of the present invention are not limited, as long as it can induce death of the aged pancreatic cancer cells, but include Quercetin, Dasatinib, Fisetin, Luteolin, Curcumin, Curcumin Analog FF24, Navitoclax (ABT263), A1331852, A1155463, Geldanamycin, Tanespimycin, Alvespimycin, Piperlongumine, FOXO4-related peptide, Nutlin3a, Cardiac glycosides (such as Ouabain, Proscillaridin A, Digoxin). However, Quercetin is preferable. However, in the present invention, pancreatic cancer cells that have been induced to aging by FGFR4 inhibitors are induced to cell death by senolytic drug. Therefore, any senolytic drug can be used as long as aging is induced in the cells.

Quercetin is a compound represented by the following formula (3):

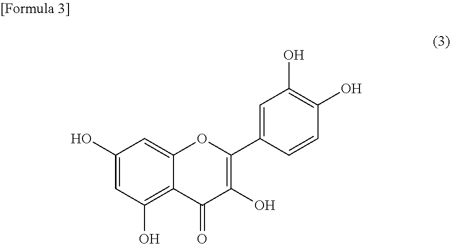 [SMILES: O=c1c(O)c(-c2ccc(O)c(O)c2)oc2cc(O)cc(O)c12]

The pharmaceutical composition of the present invention may contain, but is not limited to, 0.01 to 99% by weight, preferably 0.1 to 80% by weight, of the active ingredient. A dose of the pharmaceutical composition of the present invention may be appropriately determined in accordance with, for example, the type of each active ingredient, type of each disease, age, sex, body weight, or degree of symptom of each patient, route of administration, or the like, and the determined dosage can be administered orally or parenterally. It is desirable that the administration method, dose, administration period, administration interval, and the like, of the pharmaceutical composition to humans are determined by a controlled clinical trial. In addition, dosage form for administration of the pharmaceutical composition of the present invention is not limited to a drug medicine. That is, it can be administered as a food and drink of various form, such as a functional food, a healthy food (including drink), or an animal food stuff

The examples of solid composition for oral administration include tablets, dispersions, granules, or the like. In such solid compositions, one or more active ingredients are mixed with at least one inert excipient, such as lactose, mannitol, glucose, hydroxypropyl cellulose, microcrystalline cellulose, starch, polyvinylpyrrolidone, and/or magnesium metasilicate. The composition may contain inert additives, such as lubricants e.g., magnesium stearate, disintegrants e.g., sodium carboxymethylstatinate, stabilizer, solubilizer, etc., according to conventional methods. Tablets or pills may be coated with a sugar coating or a film of a gastric or enteric soluble substance as needed.

The examples of liquid composition for oral administration include pharmaceutically acceptable emulsion, solution, suspension, syrup, elixir, or the like, which contain commonly used inert diluent, such as purified water or ethanol. In addition to the inert diluent, the liquid composition may contain auxiliary agents such as solubilizer, wetting agent, and suspending agent, sweetening agent, flavoring agent, aromatic agent, and preservative.

The examples of injectable drug for parenteral administration include sterile aqueous or non-aqueous solution, suspension or emulsion. The examples of aqueous solvent include, distilled water or saline solution for injection. The examples of nonaqueous solvent include, propylene glycol, polyethylene glycol or vegetable oils such as olive oil, alcohols such as ethanol, or polysorbate 80 (pharmacopeia name). Such composition may further contain isotonicity agent, preservative, wetting agent, emulsifier, dispersant, stabilizer, or solubilizer. These are sterilized, for example, by filtration through a bacteria-retaining filter, the addition of a bactericidal agent, or irradiation. These can also be produced as sterile solid composition and dissolved or suspended in sterile water or sterile injectable solvent, prior to use.

### Method for Treating Pancreatic Cancer

The above pharmaceutical composition can be used for a method for treating pancreatic cancer. That is to say, this specification discloses a method of treating pancreatic cancer, characterized in that an effective amount of a fibroblast growth factor receptor 4 inhibitor and a senolytic drug is administered to a pancreatic cancer patient.

### Fibroblast Growth Factor Receptor 4 Inhibitor and Senolytic Drug for Use in Method for Treating Pancreatic Cancer

Fibroblast growth factor receptor 4 inhibitors and senolytic drugs can be used in the method for treating pancreatic cancer. That is, the present specification discloses fibroblast growth factor receptor 4 inhibitors and senolytic drugs for use in method for treating pancreatic cancer.

### Use of Fibroblast Growth Factor Receptor 4 Inhibitor and Senolytic Drug in Manufacture

The fibroblast growth factor receptor 4 inhibitors and senolytic drugs can be used in the manufacture of a pharmaceutical composition for treating prostate cancer. That is, the present specification discloses use of fibroblast growth factor receptor 4 inhibitor and senolytic drug in the manufacture of a pharmaceutical composition for treating prostate cancer.

### Function

The mechanism by which the pharmaceutical composition of the present invention can treat pancreatic cancer has not been analyzed in detail, but can be presumed to be as follows. However, the present invention is not limited by the following presumption.

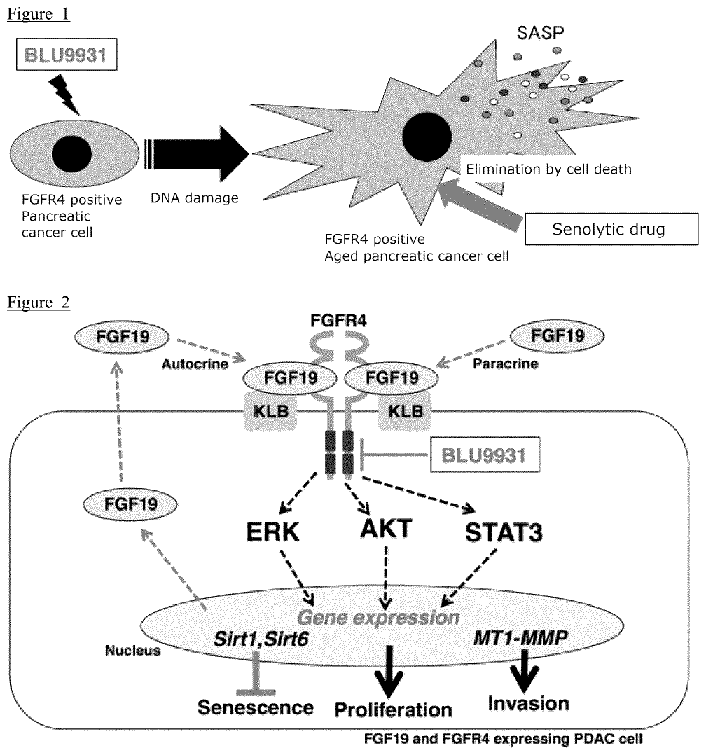

Normal pancreatic cells hardly express fibroblast growth factor receptor 4 (FGFR4). As shown in FIG. 1, some pancreatic cancer cells express FGFR4. Pancreatic cancer cells expressing FGFR4 are subjected to DNA damage by FGFR4 inhibitors such as BLU9931, and are induced aging. In addition, FGFR4 normally binds to the ligand FGF19, resulting in FGF19/FGFR4 signaling. However, as shown in FIG. 2, FGFR4 inhibitors inhibit downstream pathways such as ERK, AKT, and STAT3, which are estimated to cause decreased proliferation, inhibit invasion, and induce cellular aging. However, some pancreatic cancer cells in which cellular aging is induced may die. However, it is thought that further induction of cell death by senolytic drugs can efficiently eliminate pancreatic cancer cells.

### [2] Pharmaceutical Composition by Photoimmunotherapy

The pharmaceutical composition comprises a FGFR4 immune complex wherein an anticancer drug or a photosensitizer is bound to an antibody specific to FGFR4.

Cancer cells can be destroyed by binding immune complexes containing, for example, photosensitizer to FGFR4-positive pancreatic cancer cells in vivo and irradiating them with non-thermal red light.

### Immune Complex

The immune complex contained in the pharmaceutical composition of the present invention are complexes consisting of an anticancer drug or a photosensitizer bound to an antibody specific for FGFR4.

### Antibody

The antibody which can be used in the pharmaceutical composition of the present invention, is not particularly limited, as long as it is an antibody which specifically bind to FGFR4, or an antibody fragment having antigen binding site thereof. Preferably, it is a mouse monoclonal antibody, or a chimeric antibody thereof, or a humanized antibody (CDR-grafted antibody) thereof, or a human type antibody.

The chimeric antibody can be obtained as follows. For example, the DNA encoding the heavy chain variable region domain and light chain variable region domain of mouse antibody is linked to the DNA encoding the polypeptide of the constant region of human antibody, and it is then inserted into an expression vector, and it is introduced into the host for production. The heavy chain variable region domain and light chain variable region domain used in the chimeric antibody are not limited. The origin of the polypeptides of the constant region is not limited, as long as it is human antibody. For example, the chimeric antibody can be obtained by the heavy chain variable region domain and light chain variable region domain of mouse IgG, and the polypeptide of the constant region of human IgM or IgG.

In the humanized antibodies (CDR-grafted antibodies), the complementarity-determining regions (CDR) of, for example, human antibodies are replaced with the complementarity-determining regions (CDR) of, for example, mouse antibodies, and the complementarity-determining regions (CDR) of mouse antibodies are transplanted. Specifically, a DNA sequence designed to link the CDRs of a mouse antibody to the framework regions (FRs; framework regions) of a human antibody is synthesized by PCR from several oligonucleotides with overlapping portions at the ends. The resulting DNA is concatenated with DNA encoding the C region of the human antibody, incorporated into an expression vector, and then introduced into a host for production, to obtain the humanized antibody. An origin of polypeptides of the complementarity-determining region, the framework region, and the constant region used for humanized antibodies is not particularly limited, as long as it is human antibody. For example, the humanized antibody can be obtained from the complementarity-determining region of mouse IgG, and the framework region and the constant region of human IgM or IgG. Further, the antigen-binding fragments of humanized antibodies can be obtained from the complementarity-determining region of mouse and the human IgM or IgG framework region of human.

The human type antibody can be obtained from the transgenic animals transfected with human antibody genes, or it is a monoclonal antibody obtained by cell fusion of human antibody-producing cells with myeloma cells. As the method for obtaining the human type antibody, a technique of obtaining human antibodies by panning using a human antibody library is known, in addition to the above methods of obtaining from transgenic animals or by cell fusion of human antibody-producing cells.

For example, the variable regions of human antibodies can be expressed as single chain antibodies (scFv) on the surface of phages by phage display method, and phages that bind to the antigen can be selected. By analyzing the genes of the selected phages, it is possible to determine the DNA sequence encoding the variable regions of the human antibodies that bind to the antigen. By determining the DNA sequence of the scFv that binds to the antigen, an appropriate expression vector can be created and human antibodies can be obtained.

### Anticancer Drug

The anticancer drug used in the immune complex is not particularly limited, as long as it is effective for pancreatic cancer, but there may be mentioned tegafur, gimeracil, yrc,racii potassium, gemcitabine (Gemzar), fluorouracil [5-FU], calcium levofolinate, irinotecan, oxaliplatin, nab-paclitaxel (Abraxane), or erlotinib (Tarceva). It is also possible to bind radioisotopes to antibodies and use them as immune complexes.

### Photosensitizer

As the photosensitizer, there may be mentioned porphyrin compounds (e.g., 5-aminolevulinic acid), proline compounds (e.g., proline), bacterioproline compounds, or phthalocyanine compounds (e.g., phthalocyanine). Further, photophrin, laserphyrin, aminolevulinic acid (ALA), silicon phthalocyanine Pc4, m-tetrahydroxyphenylchlorin (mTHPC), chlorin e6 (Ce6), almera, leblanc, foscan, metvix, hexvix, photochlor, photosens, photorex, lumacan, bisonac, amfinex, Verteporfin, purlytin, ATMPn, zinc phthalocyanine (ZnPc), protoporphyrin IX (PpIX), pyropheophorbide a (PPa), pheophorbide (PhA), or the like can also be used.

For example, phthalocyanine (IR700) is chemically changed by near-infrared light (non-thermal red light) at 700 nm. That is to say, the cancer cells are damaged (expansion, destruction, or necrosis) by absorbing light energy and generating heat. Near-infrared light can also be irradiated by guiding an optical fiber near the pancreatic cancer. Furthermore, it can be designed so that the immune complex (antibody-phthalocyanine conjugate) is activated by near-infrared irradiation only when it binds to the target molecule (FGFR4).

Cancer-specific proteins released from cancer cells destroyed by the pharmaceutical composition of the present invention sensitize and proliferate cytotoxic T cells against cancer as antigens, then, the number of cytotoxic T cells attacking cancer is increased thereby. The pharmaceutical compositions of the present invention can efficiently treat pancreatic cancer by such mechanism as well.

The formulation of the pharmaceutical composition of the present invention is not particularly limited. For example, there may be mentioned, oral agents, such as powders, subtle granules, granules, tablets, capsules, suspensions, emulsions, sylups, extracts, or balls; or injections or the like, but the injections are preferable. For example, in a preparation of the injections, an aqueous solvent such as normal saline solution or Ringer solution, non-aqueous solutions such as plant oil or fatty acid ester, a tonicity agent such as glucose or sodium chloride, a solubility assisting agent, a stabilizing agent, an antiseptic agent, a suspending agent, or an emulsifying agent, may be optionally used, in addition to the active ingredient.

The pharmaceutical composition of the present invention may contain, but is not limited to, 0.01 to 99% by weight, preferably 0.1 to 80% by weight, of the active ingredient. A dose (therapeutic effective amount) of the pharmaceutical composition of the present invention may be appropriately determined in accordance with, for example, the type of each active ingredient (anticancer drug or photosensitizer), the type or stage of pancreatic cancer, age, sex, body weight, or degree of symptom of each patient, route of administration, or the like, and the determined dosage can be administered orally or parenterally.

The pharmaceutical composition of the present invention can be used in a method of treating pancreatic cancer, which is characterized by administering a therapeutically effective amount of the pharmaceutical composition to a pancreatic cancer patient.

### EXAMPLES

The present invention now will be further illustrated by, but is by no means limited to, the following Examples.

### Example 1

In this example, the expression of FGFR4 in 136 human pancreatic cancer tissues were examined. Immunostaining of pancreatic cancer tissues was performed using anti-FGFR4 antibody. In control normal pancreatic tissues, a few positive cells were found in endocrine tissues and exocrine tissues. In Table 2, the ratios of low expression (<2) and high expression (2-3) thereof are summarized by pancreatic local stage (T1-4), lymph node metastasis (N0, N1), and stage classification (stage I, II, III, IV).

Grade 1 cells are highly differentiated type and shows a morphology similar to that of normal pancreatic ducts. Grade 3 cells are poorly differentiated type and show a morphology very different from normal cells. Grade 2 cells show a morphology intermediate between that of Grade 1 and Grade 3 cells.

TABLE 2

| | Low (<2) | High (2~3) | p-value |
|---|---|---|---|
| Tumor Grade | | | 0.100 |
| Grade 1 | 15 | 8 | |
| Grade 2 | 33 | 42 | |
| Grade 3 | 19 | 12 | |
| Primary Tumor | | | <0.001 |
| T1 | 2 | 0 | |
| T2 | 27 | 8 | |
| T3 | 39 | 53 | |
| T4 | 1 | 6 | |
| Regional Lymph | | | 0.083 |
| Node | | | |
| N0 | 62 | 53 | |
| N1 | 7 | 14 | |
| Stage | | | <0.001 |
| Stage I | 27 | 2 | |
| Stage II | 34 | 45 | |
| Stage III | 7 | 16 | |
| Stage IV | 1 | 4 | |

In the pancreatic cancer tissues, the positivity rate increased as the tumor diameter increased and the stage advanced.

### Example 2

In this example, the effects of the FGFR4 inhibitor, BLU9931, on an FGFR4-positive (high expression) pancreatic cancer cell line, PK-1 cells, and a pancreatic cancer cell line with low expression of FGFR4, PK-45P cells, were examined.

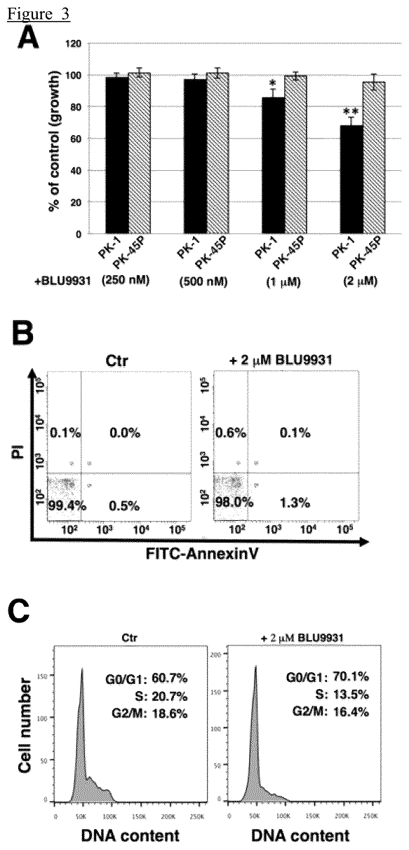

Cells were seeded at 3×10³ cells/well in 96-well plates and 250 nM, 500 nM, 1 μM, or 2 μM of BLU9931 was added. After 72 hours of incubation, the effect on cell proliferation was examined by ATP assay. As shown in FIG. 3A, in PK-1 cells with high expression of FGFR4, cell proliferation was suppressed by BLU9931 in a volume-dependent manner. On the other hand, in PK-45P cells with low FGFR4 expression, there was no effect on cell proliferation.

Further, the cytotoxicity of 2 μM of BLU9931 was examined by FACS using annexin V and PI. However, no significant effects were observed, although apoptosis, necrosis, or cell damage observed in 2% or less of the cells (FIG. 3B).

Furthermore, the effect of BLU9931 on the cell cycle was examined by FACS.

As shown in FIG. 3C, the addition of BLU9931 increased the number of cells in G0/G1 phase from 60.7% to 70.1% and decreased the number of cells in S phase from 20.7% to 13.5%. From these results, it is considered that BLU9931 slows down the transition from G1 to S phase.

In addition, the effects of the addition of BLU9931 on the expression of ERK, AKT, and STAT3 downstream of FGF19/FGFR4 signaling were examined by immunoblotting.

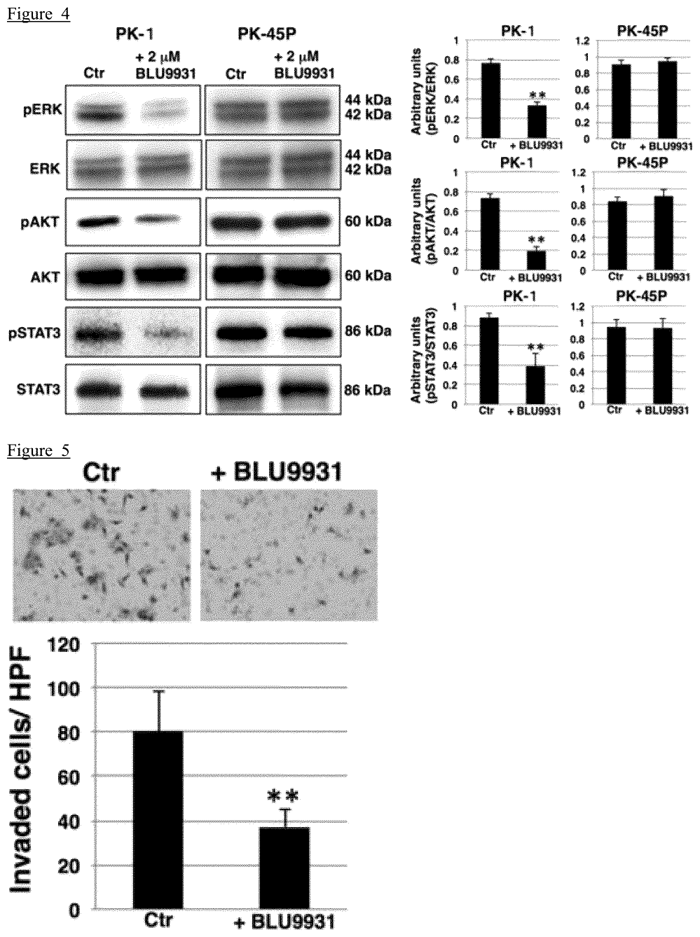

As shown in FIG. 4, in PK-1 cells, the expression of ERK, AKT, and STAT3 were decreased by the addition of BLU9931 (2μM). On the other hand, in PK-45P cells, the expression of ERK, AKT, and STAT3 were not decreased.

### Example 3

In this example, the effect of BLU9931 on cell invasion of pancreatic cancer cells was examined using a Corning Matrigel Invasion Chamber (pore size 8 μm).

1×10⁵ cells/500 μL of PK-1 cells were seeded on top of the insert. 2 μM of BLU9931 was added, and 16 hours later, cells infiltrating the lower part of the membrane were stained with a Diff-Quick staining kit. As shown in FIG. 5, the addition of BLU9931 inhibited cell invasion.

### Example 4

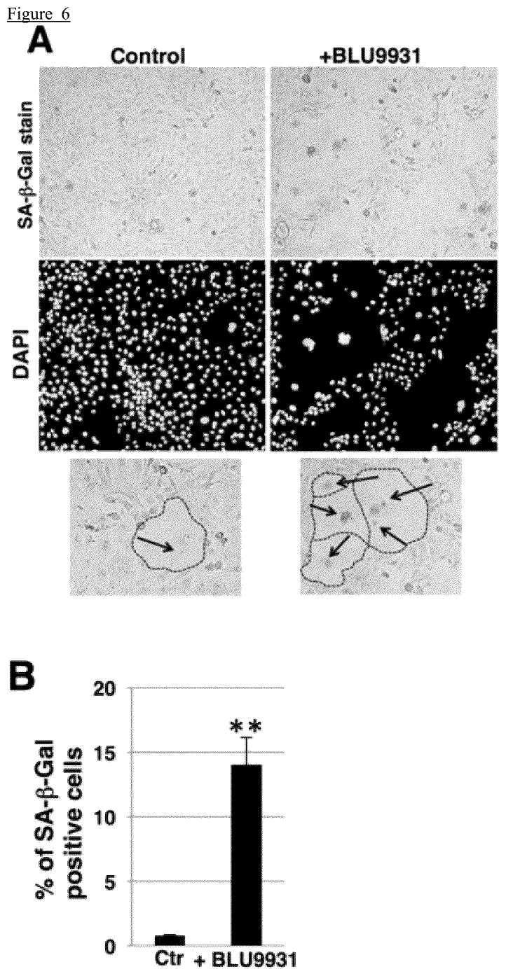

In this example, an aging of cells by BLU9931 was examined. When PK-1 cells were cultured with BLU9931 (2 μM) for 1 week, the cells became larger and flattened compared to the control cells (FIG. 6A). This morphological change was considered to be due to cellular aging. The present inventors measured the activity of senescence-associated β-galactosidase (SA-β-Gal), a marker of cellular aging.

Cells treated with BLU9931 (2 μM) were washed, treated with a fixative for 10 minutes, and incubated overnight in a staining solution. After staining, nuclear staining was performed with DAPI. As shown in FIG. 6A, the number of SA-β-Gal positive cells increased compared to the control cells.

### Example 5

In this example, DNA damage by BLU9931 was examined by phosphorylation of H2A.X, which is a marker of DNA damage.

BLU9931-treated cells were incubated with anti-γHA2.X mouse monoclonal antibody at 4° C. overnight. Cells were treated with Alexa-Fluor-conjugated secondary antibodies and nuclear staining was performed with DAPI.

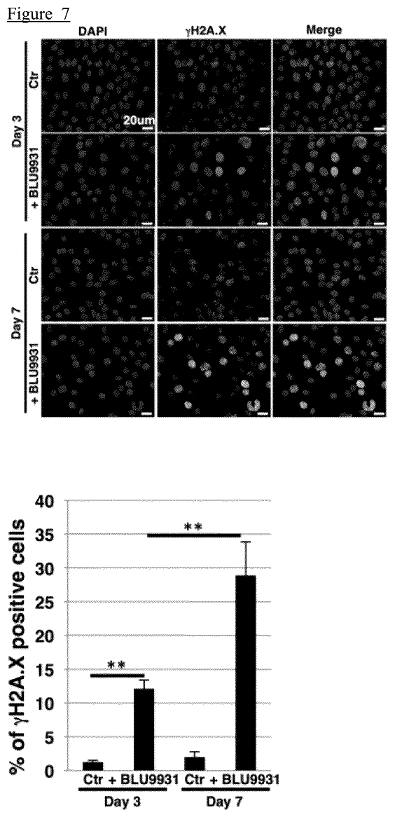

As shown in FIG. 7, DNA damage of the cells had progressed as the incubation time increased from 3 days to 7 days.

### Example 6

In this example, the survival rate of pancreatic cancer cells by FGFR4 inhibitor and senolytic drug was examined.

SP-1 cells of 3.0×10³ cells/well were cultured in the presence of BLU9931 (2 μM) for 1 week. Quercetin (12.5 μM or 25 μM) or Dasatinib (62.5nM), which were senolytic drugs, was added, and the cells were cultured for 4 days.

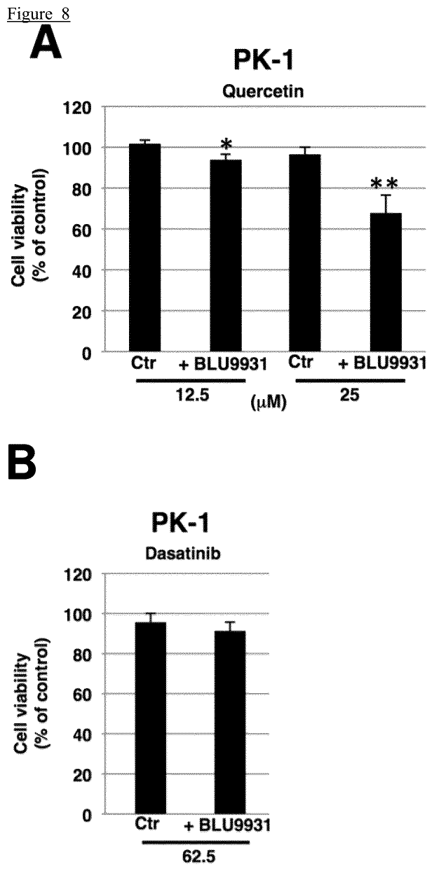

The proliferation rate was measured by ATP assay. As shown in FIG. 8, cell viability was decreased by FGFR4 inhibitor and senolytic drug.

### INDUSTRIAL APPLICABILITY

The pharmaceutical composition of the present invention can be used for treating pancreatic cancer patients.

1. A pharmaceutical composition comprising a fibroblast growth factor receptor 4 inhibitor and a senolytic drug as active ingredients.

2. The pharmaceutical composition according to claim 1, wherein the fibroblast growth factor receptor 4 inhibitor is roblitinib, BLU-554, or BLU9931.

3. The pharmaceutical composition according to claim 1 or 2, wherein the senolytic drug is quercetin.

4. The pharmaceutical composition according to claim 1, for treating pancreatic cancer.

5. A pharmaceutical composition for treating pancreatic cancer, comprising a fibroblast growth factor receptor 4 immune complex in which an anticancer agent or a photosensitive substance is conjugated to an antibody specific for fibroblast growth factor receptor 4.

6. The pharmaceutical composition for treating pancreatic cancer according to claim 5, wherein the photosensitive substance is phthalocyanine.

7. The pharmaceutical composition for treating pancreatic cancer according to claim 5, wherein the antibody is a humanized antibody.

8. A method for determining an effect of treatment of the pharmaceutical composition for treating pancreatic cancer according to claim 5.

9. A method for treating pancreatic cancer, comprising the steps of:
(a) administrating a fibroblast growth factor receptor 4 inhibitor to a pancreatic cancer patient to the pancreatic cancer cells, and
(b) administrating a senolytic drug to induce death of the senescent pancreatic cancer cells.

10. The method for treating pancreatic cancer according to claim 9, wherein the fibroblast growth factor receptor 4 inhibitor is roblitinib, BLU-554, or BLU9931.

11. The method for treating pancreatic cancer according to claim 9, wherein the senolytic drug is quercetin.

12. The method for treating pancreatic cancer according to claim 10, wherein the senolytic drug is quercetin.
